(19) United States
(12) Patent Application Publication
(10) Pub. No.: US 2013/0060103 A1
(43) Pub. Date: Mar. 7, 2013

(54) METHOD AND SYSTEM FOR USING DISTRIBUTED ELECTROMAGNETIC (EM) TISSUE(S) MONITORING

(71) Applicants: Shlomi Bergida, Tel-Aviv (IL); Amir Saroka, Tel-Aviv (IL); Dan Rappaport, Tel-Aviv (IL); Nahum Hai, Zikhron-Yaakov (IL)

(72) Inventors: Shlomi Bergida, Tel-Aviv (IL); Amir Saroka, Tel-Aviv (IL); Dan Rappaport, Tel-Aviv (IL); Nahum Hai, Zikhron-Yaakov (IL)

(73) Assignee: Sensible Medical Innovations Ltd., Natania (IL)

(21) Appl. No.: 13/697,384

(22) PCT Filed: May 12, 2011
(86) PCT No.: PCT/IL11/00377

§ 371 Date: Nov. 12, 2012

Related U.S. Application Data
(60) Provisional application No. 61/334,229, filed on May 13, 2010.

(51) Int. Cl. A61B 5/07; A61M 5/168; A61N 1/365

(52) U.S. Cl. 600/302; 607/17; 604/66

(57) ABSTRACT

A system for monitoring at least one biological tissue of a patient during a period of at least 24 hours. The system comprises an implantable intrabody probe and an extrabody probe which propagate an electromagnetic (EM) signal, using an antenna, via at least one tissue therebetween, in a plurality of sessions during a period of at least 24 hours, a processing unit which analyses the EM signal to detect a change in at least one biological parameter of the at least one tissue, and an output unit which outputs the change.

### RELATED APPLICATION

This application claims priority from U.S. Patent Application No. 61/334,229, filed on May 13, 2010. The contents of all of the above documents are incorporated by reference as if fully set forth herein.

### FIELD AND BACKGROUND OF THE INVENTION

The present invention, in some embodiments thereof, relates to biological tissues monitoring and, more particularly, but not exclusively, to methods and system of using EM measurements for monitoring biological tissues.

In monitoring systems, which are based on analysis of EM signals, EM radiation signal is delivered into the body, propagates therethrough and/or reflected therefrom, and then intercepted and evaluated.

During the last years various monitoring systems have been developed. For example, U.S. Patent Application Pub. No. 2010/0056907, filed on Aug. 20, 2009, describes a method for monitoring at least one cardiac tissue. The method comprises a) intercepting a plurality of reflections of an electromagnetic (EM) radiation reflected from at least one cardiac tissue of a patient in a plurality of EM radiation sessions, b) computing a mechanical tracing indicative of at least one mechanical property of said at least one cardiac tissue according to said plurality of reflections, c) analyzing said mechanical tracing so as to detect a presence or an absence of a physiological condition, and d) outputting said analysis.

Another example is described in U.S. Patent Application Pub. No. 2010/0256462, filed on Sep. 4, 2008, which teaches a method for monitoring thoracic tissue. The method comprises intercepting reflections of electromagnetic (EM) radiation reflected from thoracic tissue of a patient in radiation sessions during a period of at least 24 hours, detecting a change of a dielectric coefficient of the thoracic tissue by analyzing respective the reflections, and outputting a notification indicating the change. The reflections are changed as an outcome of thoracic movements which occur during the period.

As in such systems and methods EM signals have to pass through the area of interest for providing data pertaining to specific biological parameters, the EM signals often experiences severe attenuations, distortions and delays. The quality of the EM signals may be increased if the sensors are placed in an optimal position in relation to the monitored biological tissue. In order to extract trends of biological parameter(s), it is important to fixate the sensors to capture EM radiation n a plurality of intervals over a relatively long period. The sensors have to be placed properly so as to reduce the attenuation and/or the delay that the EM signal experiences.

### SUMMARY OF THE INVENTION

According to some embodiments of the present invention a system for monitoring at least one biological tissue of a patient during a period of at least 24 hours. The system comprises an implantable intrabody probe and an extrabody probe which propagate an electromagnetic (EM) signal, using an antenna, via at least one tissue therebetween, in a plurality of sessions during a period of at least 24 hours, a processing unit which analyses the EM signal to detect a change in at least one biological parameter of the at least one tissue, and an output unit which outputs the change.

Optionally, the extrabody probe delivers and intercepts the EM signal.

Optionally, the extrabody probe delivers the EM signal and the intrabody probe intercepts the EM signal.

Optionally, the processing unit analyses the EM signal to detect a change in a fluid level of the at least one tissue.

Optionally, the processing unit is part of the implantable intrabody probe; the implantable intrabody probe comprises a communication interface to transmit data pertaining to the analyzed EM signal to at least one of the extrabody probe and an extrabody patient management unit.

Optionally, the processing unit is part of at least one of the extrabody probe, an extrabody patient management unit, and an implantable medical device (IMD).

Optionally, the implantable intrabody probe is integrated with an implantable medical device (IMD).

Optionally, the implantable intrabody probe is housed in a housing made of a biocompatible material which minimally attenuates EM signals.

Optionally, the system further comprises a communication interface for receiving at least one parameter from an implantable medical device (IMD); the processing unit calculates the change according to a combination of an analysis of the EM signal and the at least one parameter.

Optionally, the system further comprises a communication interface for receiving at least one parameter from an implantable medical device (IMD); and being operated according to the at least one parameter.

Optionally, the system further comprises a communication interface for forwarding data related to the change to an implantable medical device (IMD) so as to allow regulation of operation of the IMD.

More optionally, the change is a cardiac ejection fraction change, and wherein the IMD is a pacemaker device, and wherein the regulation comprises adjusting the pacing parameters of the pacemaker according to the cardiac ejection fraction change.

More optionally, the processing unit calculates cardiac output according to the change, the regulating comprises adjusting the pacing parameters of a pace making element according to the cardiac output.

More optionally, the IMD comprises a drug releasing element, the IMD adjusting the releasing pace of the drug releasing element according to the change.

More optionally, the change is a rate of change of the pressure in the heart of the patient, the IMD adjusting the pacing parameters of a pace making element according to the rate.

Optionally, the implantable intrabody probe comprises an active element which performs at least one of intensifying, regenerating, and manipulating the EM signal.

Optionally, at least one of the intrabody and extrabody probes comprises an additional sensor for gathering data related to the physical condition of the patient; the processing unit combines the change and the gathered data to determine a biological parameter.

Optionally, the system further comprises a communication interface for receiving pressure value from a pulmonary arterial pressure (PAP) device implanted into the patient; the processing unit calculates the change according to a combination of an analysis of the EM signal and the pressure value.

Optionally, at least one of the implantable intrabody probe and the extrabody probe propagate a plurality of EM signals, using an antenna, via at least one tissue therebetween in a plurality of sessions during a monitoring period of at least 24 hours.

Optionally, at least one of the implantable intrabody probe and the extrabody probe comprises a communication interface to transmit data pertaining to the EM signal to an external management unit that comprises the processing unit.

Optionally, the processing unit analyses the EM signal to detect a trend of at least one dielectric related property of the at least one tissue.

Optionally, the extrabody probe is integrated into a wearable element.

Optionally, the extrabody probe is integrated into at least one of a wall, a mattress, a handheld device, a Smartphone and a piece of furniture.

Optionally, the EM signal is a radio frequency (RF) signal.

Optionally, the EM signal is a microwave (MW) signal.

Optionally, the intrabody probe transmits the EM signal; further comprising an amplitude detector for sampling the amplitude of the EM signal and a communication interface for transmitting the sampled amplitude to the extrabody probe.

Optionally, the intrabody probe transmits the EM signal; further comprising a module for coordinating the phase of the EM signal according to the phase of a different EM signal that is received by it.

Optionally, the intrabody probe transmits a plurality of EM signals toward a plurality of extrabody probes including the extrabody probe, and wherein the change is calculated according to at least one differential measurement derived from signals derived by the probes.

Optionally, the extrabody probe transmits the EM signal toward the intrabody probe; the intrabody probe comprises a reflector for reflecting the EM signal toward at least one of the extrabody probe and another extrabody probe.

Optionally, the intrabody probe comprises an inductive coil for intercepting an inductive charging field to charge the intrabody probe.

More optionally, the extrabody probe comprises a power supply element circuitry for generating the inductive charging field.

Optionally, the intrabody probe shares a power source with an implantable medical device (IMD) in the body of the patient.

According to some embodiments of the present invention a method for monitoring at least one biological tissue of a patient. The method comprises implanting an implantable intrabody probe in an intrabody area of a patient, positioning an extrabody probe in proximity to a skin area of the patient, propagating an electromagnetic (EM) signal via at least one tissue between the intrabody area and the skin area and intercepting EM signal, the propagating and intercepting being performed by the implantable intrabody probe and the extrabody probe, analyzing the propagated EM signal to detect a change in at least one biological parameter of the at least one tissue, and outputting the change.

Optionally, the implantable intrabody probe is implanted between a muscle layer and a fat layer of the patient.

Optionally, the implantable intrabody probe is implanted within the chest cavity and directed to transmit the EM signal via the lung toward the extrabody probe.

According to some embodiments of the present invention an implantable intrabody probe for monitoring at least one biological tissue of a patient during a period of at least 24 hours. The implantable intrabody probe comprises a transmitter which propagates an electromagnetic (EM) signal, using an antenna, via at least one tissue, a receiver which intercepts a reflection of the EM signal from the at least one tissue, a processing unit which analyses the reflection to detect a change in at least one biological parameter of the at least one tissue, and a communication unit which transfers a message, based on the change, to an extrabody unit.

According to some embodiments of the present invention a system for monitoring at least one biological tissue of a patient during a period of at least 24 hours. The system comprises an implantable reflecting element, an extrabody probe which captures an electromagnetic (EM) signal, using an antenna, which is propagated via at least one tissue between the extrabody probe and the implantable reflecting element and captures a reflection of the EM signal from the implantable reflecting element, and a processing unit which analyses the reflection to detect a change in at least one biological parameter of the at least one tissue.

According to some embodiments of the present invention a device for monitoring at least one biological tissue of a patient. The device comprises an extrabody probe which captures an electromagnetic (EM) signal, using an antenna, that is transmitted via at least one intrabody tissue, a communication interface for receiving cardiac data from an implantable medical device (IMD), and a processing unit which analyses the reflection and calculates at least one biological parameter of the at least one intrabody tissue according to a combination of an analysis of the EM signal and the cardiac data.

Optionally, the cardiac data pacing parameters of a pace making element.

According to some embodiments of the present invention a device for monitoring at least one biological tissue of a patient. The device comprises an extrabody probe which captures an electromagnetic (EM) signal, using an antenna, that is transmitted via at least one intrabody tissue, a processing unit which analyses the EM signal and calculates at least one biological parameter of the at least one intrabody tissue accordingly, and a communication interface for transmitting at least one of the at least one biological parameter and instructions calculated based on the at least one biological parameter to an implantable medical device (IMD).

Optionally, the at least one biological parameter comprises a member selected from a group consisting of a cardiac ejection fraction change, rate of pressure rise, and a cardiac output.

According to some embodiments of the present invention a method for monitoring at least one biological tissue of a patient. The method comprises implanting an implantable intrabody probe in an intrabody area of a patient, propagating an electromagnetic (EM) signal via at least one tissue, intercepting EM signal, analyzing the propagated EM signal to detect a change in at least one biological parameter of the at least one tissue, and wirelessly transferring a message, based on the change, to an extrabody unit.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of a monitoring system for monitoring biological parameter(s) of one or more biological tissues according to an electromagnetic (EM) signal that is propagated between an implantable intrabody probe(s) and an extrabody probe(s), according to some embodiments of the present invention;

FIG. 2 is a schematic illustration of a set of components, some optional, of an exemplary extrabody probe, according to some embodiments of the present invention;

FIGS. 3A-3C are schematic illustrations on an axial thoracic plane of a patient monitored by the monitoring system 100, according to some embodiments of the present invention;

FIG. 4 is a schematic illustration of an intrabody probe of a monitoring system that transmits data pertaining to measurements directly to a patient management unit (not via an extrabody probe), according to some embodiments of the present invention;

FIG. 5 is a schematic illustration of a set of components, some optional, of an exemplary intrabody probe, according to some embodiments of the present invention;

FIG. 6 is a schematic illustration of an intrabody probe of a monitoring system that transmits data pertaining to measurements directly to an interrogator device, according to some embodiments of the present invention;

FIG. 7 is a schematic illustration of a network of interrogator devices, according to some embodiments of the present invention;

FIG. 8 is a sectional schematic illustration of a probe which may be an intrabody or extrabody probe, according to some embodiments of the present invention; and

FIG. 9 is a flowchart of a method for monitoring a bodily tissue, for example thoracic tissue, using a monitoring system, for example as depicted in FIG. 1, according to some embodiments of the present invention.

### DESCRIPTION OF EMBODIMENTS OF THE INVENTION

The present invention, in some embodiments thereof, relates to biological tissues monitoring and, more particularly, but not exclusively, to methods and system of using EM measurements for monitoring biological tissues.

According to some embodiments of the present invention, there is provided a system for monitoring one or more biological tissues of a patient, optionally during a period of more than 24 hours. The system includes an implantable intrabody probe and an extrabody probe which propagate electromagnetic (EM) signal(s), using one or more antennas, via one or more biological tissues therebetween. For example, the electromagnetic (EM) signals, which for brevity may be referred to herein as an EM signal, may be transmitted by one probe and intercepted and/or processed by or reflected from the other probe. The implantable intrabody probe may be a designated probe or a component that is integrated with or into another implantable medical device (IMD), such as a pacemaker, an implanted electrode device or a pulmonary arterial pressure (PAP) device. The system further includes a processing unit which analyses the EM signal(s) to detect a change in one or more biological parameters of the tissue(s). The processing unit may be integrated with any of the probes, shared with the IMD, and/or part of a patient management unit that communicates with any of the probes. The processing unit is connected to an output unit which outputs the change and/or instructions which are calculated based thereupon. As further described below, the system allows reducing artifacts which are associated with monitoring probes and increasing the compliance of patients for example due to a relatively easy positioning of extrabody probe.

Optionally, the monitoring is performed by measuring the dielectric related properties of tissues which are affected by the EM signals during session held in a period of 24 hours or more, for example fluid content or volume changes. The system is suitable for continuous, optionally wearable, mode of use, as well as for intermittent measurements.

Each probe may comprise of multiple elements, some or all transmitting and some or all receiving, where some might be receiving and transmitting interchangeably (change over time) in the same frequencies.

According to some embodiments of the present invention, there is provided a method for monitoring one or more biological tissues of a patient, optionally during a period of more than 24 hours. The method is based on an implantable intrabody probe which is implanted in an intrabody lumen of a patient, for example between the fat and muscle layers optionally and on an extrabody probe which is positioned in proximity to the skin of the patient, for example attached thereto. After the implantation and positioning an EM signal is propagated, for example transferred, via the one or more tissues between the intrabody lumen and the skin. Now, the propagated EM signal is analyzed to detect a change in one or more biological parameters of the tissues. This allows outputting the change, for example presenting it to a user or generating instructions for operating another IMD based thereupon.

According to some embodiments of the present invention, there is provided an implantable intrabody probe for monitoring at least one biological tissue of a patient during a period of at least 24 hours. The implantable intrabody probe includes a transmitter which propagates an EM signal, using an antenna, via one or more tissues, a receiver which intercepts a reflection of said EM signal from said the tissue(s), a processing unit which analyses the reflection to detect a change in one or more biological parameter(s) of the tissue(s) and a communication unit which transfers a message, based on said change, to an extrabody unit, such as a patient management unit or an interrogator, such as a wearable interrogator or a stationary interrogator.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

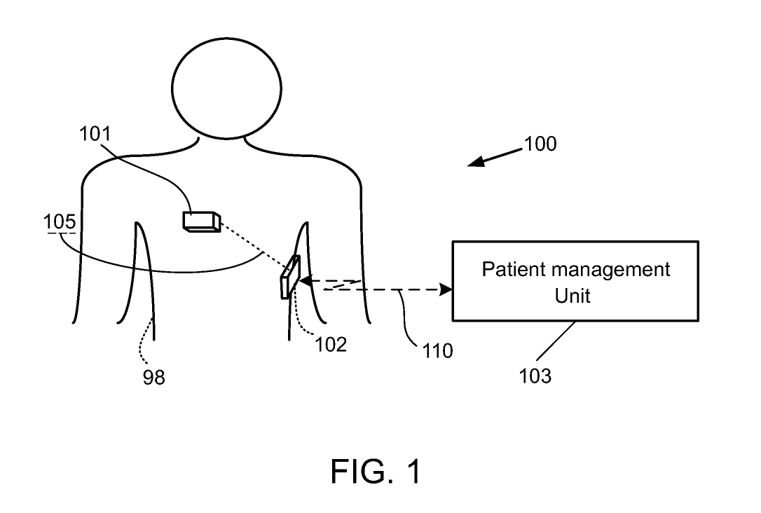

Reference is now made to FIG. 1, which is a schematic illustration of a monitoring system 100 for monitoring biological parameter(s) of one or more biological tissues according to an electromagnetic (EM) signal that is propagated between an implantable intrabody element 101, such as a reflector, an EM signal modulating element, and an intrabody probe, which may be part of a pacemaker or any other implant, and an extrabody probe 102, which is optionally placed on or in proximity to the skin of a patient, according to some embodiments of the present invention. For brevity, an intrabody element, a reflector, and an EM signal modulating element may be referred to herein interchangeably.

Biological parameter may be determined based on a combination between the dielectric related properties and additional data that is acquired using the EM transducers, such as breathing rate and/or depth and heart rate. Optionally, biological parameter may be determined based on a combination between the dielectric related properties user related data from external sources and/or sensors.

In use, the probes 101, 102 are optionally placed so that most of the energy of an EM signal, for example the radiation or power density, of the transmitted EM signal passes via a region of interest, for example an organ of interest, such as the lungs.

Optionally, one of the probes 101, 102 includes a transmitter and while the other includes a receiver. For example, the implantable intrabody element 101 may be a probe that includes an EM receiver, for example a radio frequency (RF) and/or a microwave (MW) receiver and the extrabody probe 102 which is optionally RF and/or MW transmitter. Optionally, both of probes 102, 101 include transceivers (as a single module or as separate receiver and transmitter). In this embodiment, EM signals may be propagated in both directions, from the extrabody probe 102 to the implantable intrabody element 101 and vice versa. Alternatively, only one probe is sending and receiving the EM radiation. It should be noted that tough only one extrabody probe 102 and one intrabody probe 101 are described herein, any number of extrabody probes 102 and/or intrabody probes 101 may be used, interchangeably or simultaneously. The probes may be adapted to transmit and/or intercept a plurality of EM signals in a plurality of continuous or intermittent sessions during a monitoring period which is longer than 1, 2, 4, 8, 12, 16, 20 and 24 hours, days, weeks, months, and/or years, in which the patient may be ambulatory and/or in a monitoring position. Numeral 105 depicts the pass of an exemplary EM signal that is propagated between the probes. Optionally, the extrabody probe 102 communicates with a patient management unit 103, optionally via a wireless connection 110, for example as further described below.

As the location of the implantable intrabody element 101 is fixated in the body 98 of the patient, the sensitivity to environment noises and/or currents and/or to sensor movement artifacts are reduced in relation to extrabody EM signal monitoring devices.

Moreover, the fixation of the implantable intrabody element 101 makes the monitoring system 100 less prone to unwanted movements in relation to the body of the patient and/or the monitored region of interest, and/or less prone to other movements and/or movement effects. Movement effects may refer to various movements of the monitored patient, or of the at least one external part of the monitoring device, or of organs of the monitored patient and the like. Said movements may be for example thoracic movement, organ movement, antenna movement, change of posture or position (whether voluntary or nonvoluntary changes of positions of the monitored patient) related movement, activity related movement, effects of internal physiological activities, effects of external physiological activities, irregularities such as noises, disturbances and/or interferences. Such movements and/or movement effects may have an adverse influence on the extraction of useful information from EM signals. Moreover, the implantable intrabody element 101 is less or not affected by challenges of coupling a sensor to the body of a patient, for example lose coupling to the body which results in reducing the transmission energy and/or altering phase and/or amplitude of the radiated energy.

As after the implantable intrabody element 101 has been implanted, it does not have to be positioned, repositioned and/or registered. Positioning, repositioning and/or registration are procedure which takes substantially amount of time and reduce patient compliance. In such a manner, positioning, repositioning and/or registration artifacts are avoided. It should be noted that as the implantable intrabody element 101 may be used to replace a probe that is set to be attached to the back of the patient, avoiding repositioning and/or registration artifacts may be important to the compliance of the patient and/or the robustness of the signal analysis. Attaching a probe to the back of a patient, for example when monitoring a tumor on the back, is a task that is hard to perform without support. The implantation of the intrabody element 101 allows the user to avoid this process and only to take care of the positioning of the extrabody probe 102. This allows self-placement of the monitoring system 100 and ease of measurement increases the adherence of patients to use the monitoring system 100 to perform measurements. Said adherence for example in CHF patients, allows monitoring the accumulation of fluids in the patient's lungs more accurately and can prevent hospitalization as the patient can take medications before her situations deteriorates.

Optionally, the transmissions of the intrabody element 101 and/or reflections thereof serve as a reference signal for assisting in the positioning and/or repositioning of the extrabody probe 102.

In addition, the implantation of the implantable intrabody element 101 of the monitoring system 100, instead of placing a probe in an opposite side to the location of the extrabody probe 102, reduces parasitic EM and/or electrical fields that traverse from a transmitting probe to a receiving probe for example through the skin and/or through the fat layer which may also be known as crosstalk and hold little or no relevant information. By using intrabody probe one can achieve more localization of the EM radiation around the organ of interest.

At least one of the probes 101, 102 includes or communicates with a processing unit which analyses the propagated EM signals to detect a change one or more biological parameters of the one or more tissues which are placed between the probes 101, 102, for example as further described below. The processing unit may be located in an external patient management unit 103, for example as exemplified in FIG. 1, or in any of the probes. Optionally, is connected to an output unit which outputs, for example transmits or presents, the detected change. For example, the output unit includes a transmitter, for wirelessly transmitting the change to a central monitoring unit. In another example, the output unit includes a screen for presenting the calculated change. Additionally or alternatively, the change is optionally recorded in a repository, such as a flash memory unit. It should be noted, that the term processing unit may mean herein a local processing unit, a distributed processing unit, and/or a remote processing unit. For example a remote processing unit may be a processing unit of an implantable medical device (IMD) that communicates with the monitoring system 100 or an integrated processing unit shared by the IMD and the intrabody element 101, or a processing unit that is used by the extrabody probe 102. In an embodiment in which the processing unit is remote, the data which is forwarded to the processing unit may be transmitted for remote processing by the remote processing unit. Optionally, the processing unit may include algorithms that mitigate artifacts and noise that may reduce the quality of the measurements performed by the apparatus. For example these algorithms may include algorithms used to mitigate effects of internal and external body movements and/or posture changes effects, for example registration based algorithms for example as detailed in international patent application pub. No WO 2010/100649, International patent application pub. No WO 2009/031150, and/or International patent application pub. No 2009/031149. Optionally, the intrabody element 101 is a probe that may perform only some of the required processing, for example choosing a proper time to perform the analysis of the measurements, and the extrabody probe 102 performs the other processing activities, resulting in calculating the respective biological parameter.

The analysis of the propagated EM signals is optionally based on dielectric related properties and/or dielectric related property changes of the tissue(s), for example as described in international patent application pub. No WO 2010/100649, International patent application pub. No WO 2009/031150, and/or International patent application pub. No 2009/031149, which are incorporated herein by reference. As used herein, a biological parameter means any one or more values of biological indicators which reflect a property of one or more organs and/or tissues, for example fluid level in a tissue, the size and/or type of a tumor, mechanical movements of an organ, dielectric related properties of a tissue and changes thereof and the like. Optionally, a biological parameter may be a trend for example the values of one or more measured biological parameters over time. As used herein, a dielectric related property of a specific volume, organ, or tissue includes one or more of magnetic permeability, electric permittivity and conductivity of the composite material within a specific volume. Such a dielectric related property may be affected by presence or distribution of fluid, concentration of substances, such as salts, glucose, in the fluid in the internal tissue and/or organ, the ratio of fibrotic tissue, a concentration of inflammatory substance in the fluid in the internal tissue and/or organ and physical configuration of organs or tissues of different properties in the volume measured. As used herein, a dielectric related property change is optionally a change that is indicative of a change in one or more dielectric related properties and/or in the configuration of intrabody tissues or in an intrabody lumen between tissues. For example, in case of a fluid change in the intrabody lumen, such as when blood fills the tissue parenchyma, a change in the dielectric coefficient of the region is expected. In another example, an ischemic region within a tissue may change its dielectric related properties to fibrotic tissue reflected by lower dielectric coefficient. In another example, a region may change dielectric related properties as a result of a cancerous tumor within a region growing in size or becoming more vascularized.

Optionally, the processing unit calculates a dielectric related property or a dielectric related property change by analyzing the changes in the intercepted propagated EM signals during a number of EM signal transmission sessions and over a monitoring period. Optionally, the EM signal is a radio frequency (RF) signal, for example at a frequency range as detailed below. Optionally, the EM signal is a microwave (MW) signal, for example at a frequency range as detailed below.

Optionally, the implantable intrabody element 101 is a probe that comprises an external wraparound, such as a capsule, that binds its components and facilitates the implantation. In use, the implantable intrabody element 101 may be subcutaneously implanted. Alternatively, the implantable intrabody element 101 may be implanted in a sub-muscular area. Such implantations are usually performed in an operation that requires only local anesthesia. The implantable intrabody element 101 may also be implanted is a more invasive operation, for example implanted deeper into the body of the patient next to the organ or tissue monitored.

According to some embodiments of the present invention, the implantable intrabody element 101 is designed to be implanted within the thorax and the extrabody probe 102 is set to be placed on the surface of the thorax, for example on the front of the thorax or at the lateral side thereof, below the armpit on or about the mid axillary line, or in proximity to the lower portion of the pleural cavity, above the diaphragm.

Other embodiments may include implantation near other regions of interest, for example in case the characteristics of the heart are measured a possible position of implantation is above the heart, on the axis between the nipples, left to the sternum. For chronic heart failure the left chambers of the heart or immediately attaching vessels may be preferred locations. Other places may also be acceptable locations. In case the region of interest is a tumor, for example a bone tumor, an implantation location proximate to the on the bone is a possible location. In the case where the monitored tissue is a tumor in the abdomen the intrabody probe may be inserted on the side of the tumor opposite the external element.

In another example, the intrabody element 101 is implanted subcutaneously or beneath the fat layer, above the muscle layer in the back of the patient and the extrabody probe 102 is placed substantially in parallel to the lower portion of the pleural cavity, above the diaphragm. The extrabody probe 102 may be positioned in several other positions on the body of the patient

The extrabody probe 102 may be attached to the patient's body using a sticker or a designated attachment unit and/or placement unit, for example as described in International Patent Applications Numbers IL2008/001198 and IL2008/001199, filed on Sep. 4, 2008 which are incorporated herein by reference. The extrabody probe 102 may be integrated into a garment, a strap, a vest a piece of cloth, and/or into another device that is positioned on the patient's body, such as music player and/or a pulsometer. The extrabody probe 102 may be optionally attached to the patient's skin using an adhesive. The extrabody probe 102 may be attached for the entire duration of a monitoring period or it may be removed and replaced once or several times during that period.

The extrabody probe 102 may be placed in proximity to the body of the patient, for example positioned in or on a mattress, a bed frame, on a wall in the patient's home and/or a chair. For example the extrabody probe 102 may be a handheld device that is held interchangeably in proximity to the body of the patient or a device incorporated into a patient's bed. In such an embodiment, the monitored patient has just to stand, sit, or lie next to the extrabody probe 102, and to initiate the monitoring session without having to wear or attach the extrabody probe 102. Optionally, different extrabody probes 102 may be used interchangeably, for example based on the proximity of the patient thereto. In some embodiments, for example where the extrabody probe is positioned close to the intrabody probe, for example when the intrabody probe is implanted subcutaneously, the extrabody probe may enhance the isolation of the intrabody probe from the environment external to the body, and/or shield an intrabody probe from interference to and from EM radiation external to the patient's body. Such isolation and/or shielding may be done by using materials and configuration for the external part as described in application No. 61/090,356—ELECTROMAGNETIC EM PROBES, METHODS FOR FABRICATION THEREOF, AND SYSTEMS WHICH USE SUCH ELECTROMAGNETIC EM PROBES, for example in an external part or in an interrogator as explained below that is placed over the intrabody probe's position.

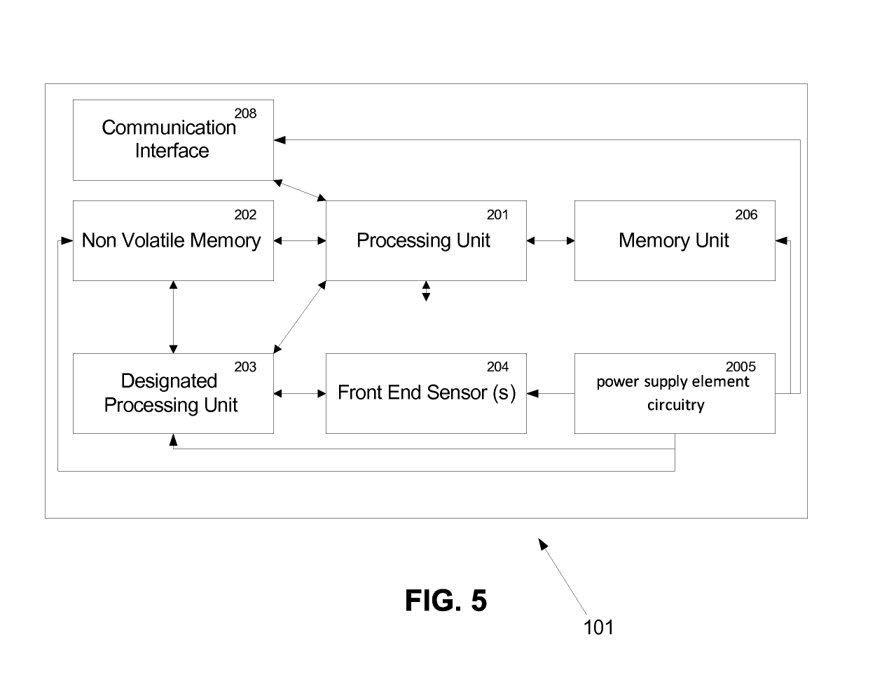

According to some embodiments of the present invention, the extrabody probe 102 includes one or more front end sensors 204 with a transmitter and optionally an antenna for transmitting the EM signal toward a receiver which is installed in the intrabody element 101. The receiver intercepts the EM signal and analyzes it, at least partly, using the processing unit that is optionally installed therein. Now, the processing unit forwards the processed data to the communication unit (i.e. FIG. 5) that transmits the processed data to the patient management unit 103. For example, the processing unit 201 (i.e. FIG. 5) calculates a biological parameter, such as a thoracic tissue fluid level, or one or more dielectric related parameters or changes, as described above, and transmits the processed data to the patient management unit 103. Optionally, a receiver that is installed at the extrabody probe 102 receives the transmitted processed data and optionally forwards it to the patient management unit 103. Optionally, the extrabody patient management unit 103 further processes and/or stores the received processed data. Optionally, additional information pertaining to the monitored patient is sent to the extrabody patient management unit 103, for example trends of biological parameters, for instance as calculated by the processing unit, extrabody and/or intrabody probe functioning status, for example battery status, memory status, faults or malfunctions, and the like.

Optionally, the functioning of the implantable intrabody element 101 is a probe that is controlled by instructions messages, optionally sent to it by extrabody probe 102 including download parameters, download software or firmware updates, change of parameters instructions, parameters of monitoring algorithms, other functional algorithms, and/or initiating measurements received via the transmitter. Optionally, the instructions messages instruct the activation and/or deactivation of the implantable intrabody element 101, allow a remote access to low level memory content, initiate self tests, resetting and the like.

According to some embodiments of the present invention, the implantable intrabody element 101 is integrated or a part of an implantable medical device (IMD), as outline above. Optionally, such an intrabody element 101 may share a resource with other components and/or functions of the IMD, for example the memory unit, the power supply, the processing unit, and/or the communication unit. Said sharing may be for example by using the computing and/or processing capabilities of the IMD to perform processing functions, for example the algorithmic functions or calculation of a biological parameter such as fluid content in a tissue based on the dielectric related properties analyzed, thus integrating the processing and/or computing device of the IMD with the intrabody probe. By sharing resources, such as communication capabilities and/or energy storage and/or energizing solutions multiple units are avoided, energy, space and cost are saved. Said integration may be mechanically and/or electronically integration or integration achieved via communication into another implanted device.

It should be noted that integration, as described herein, does not necessarily mean physical integration. For example, the IMD and the intrabody element 101 may communicate with each other via a wired and/or a wireless connection. For example, sensed parameters, such as the heart rate may be sent from the IMD to the intrabody element 101 and/or to the extrabody probe which uses it for example to detect the activity level of the patient.

It should be noted that when the implantable intrabody element 101 is integrated or a part of the IMD, a single implantation procedure is performed on a patient to implant both devices instead of two different procedures.

Information derived from the monitoring system 100 may be used to regulate the activity of an IMD that communicates or integrates the implantable intrabody element 101 or vice versa.

According to some embodiments of the present invention, the IMD is a pacemaker, for example an implanted cardiac device (ICD) or an implanted cardiac resynchronization device (CRT-D). In such an analysis of the data which is monitored by the monitoring system 100 may be used to regulate the pace, the rhythm, and/or the power of the IMD. For example, the regulation is performed according to one or more biological parameters, fluid level, dielectric related properties, dielectric related property changes, and/or mechanical movement of an organ as detailed below. For example, data obtained from the monitoring apparatus, for example biological parameter, fluid level, mechanical movement of an organ as detailed below or other data can be used in the IMD's algorithm, for example the algorithm that determines the pacing actions of a CRT-D, to influence the actions of said IMD. In such an embodiment, the processing unit or processing device inside the IMD may integrate the data from the intrabody probe of the monitoring apparatus into its active functions for example to regulate the pacing protocol.

In such embodiments, the pacemaker calculates pacing actions according to the data which is monitored by the monitoring system 100. For example, the monitoring system 100 may calculate current cardiac ejection fraction (end diastolic volume (EDV) divided by end systolic volume (ESV)) according to the timing of cardiac intervals and the pacemaker may adjust pacing parameters accordingly to optimize the cardiac ejection fraction.

Cardiac intervals that allow calculating the ejection fraction may be related to mechanical movement of the heart which may be assessed by the monitoring system 100. The calculating of the mechanical movement of the heart may be performed as described in U.S. patent application Ser. No. 12/544,314 METHODS AND DEVICES OF CARDIAC TISSUE MONITORING AND ANALYSIS filed on 20 Aug. 2009 which is incorporated herein by reference. For example, if the ejection fraction is measured to be ⅓, than a change of the pacing parameters may be performed to result in an ejection fraction of ½.

In another example, the monitoring system 100 may calculate cardiac output and the pacemaker may adjust pacing parameters accordingly. In another embodiment, the rate of change of the pressure in the heart may be calculated by the monitoring system 100, for example by calculating the rate of rise of left ventricular pressure (dP/dt) by measuring the mechanical movement of the heart by the monitoring system 100. This allows adjusting the pacemaker accordingly.

In another example, the IMD may be a Ventricular Assist Device (VAD) and its activity may be regulated by the monitoring system 100, for example by optimizing the heart's capacity according to the cardiac movement and/or ejection fraction as measured by the monitoring system 100.

Optionally, the monitoring system 100 may be used with an IMD, such as a pulmonary arterial pressure (PAP) device, or an implantable pressure sensor, or with a non-implantable IMD for example a Swan-Ganz catheter, to obtain pulmonary edema status. In such an embodiment, the IMD provides a pressure value that is indicative of edema development chances in a target area and the monitoring system 100 provides the current level of fluid in a target area. By combining both the current status (fluid level) and the anticipated status (edema development chances) a comprehensive review of the tendency of the target area is received.

Optionally, measurements of both the IMD and the implantable intrabody element 101 are transmitted to the extrabody probe 102. In such a manner, a common communication channel is used for both measurement units, saving energy and reducing radiation.

According to some embodiments of the present invention, the IMD is a drug releasing device. In such embodiments the drug releasing pace, amount, and/or timing may be regulated according to the biological parameters which are monitored by the monitoring system 100. The releasing may be adjusted as detailed below and explained in international patent application pub. No WO 2010/100649, International patent application pub. No WO 2009/031150, and/or International patent application pub. No 2009/031149, which are incorporated herein by reference.

It should be noted that as the implantable intrabody element 101 and the IMD are located at the same place, the measurements thereof are locally synchronized, for example in relation to the posture and/or the activity level of the patient. This facilitates calculating a trend or a conclusion based on the combination of measurements.

Optionally, the implantable intrabody element 101 is implanted in proximity to a monitored target area, for example an area that confines one or more organs, such as the heart. In such a manner, attenuation, disturbance and/or interference introduced by other intrabody areas, for example the skin tissue, the fat tissue, the muscle tissue, and/or the bone tissue that may contain little or no information regarding the biological parameter monitored, are reduced in relation to a monitoring that is based only on the measurements of extrabody probe(s), and such embodiment enables to achieve more localization of the EM radiation through the organ of interest. When a single extrabody probe is used EM signals are propagated toward and from the target area. When a number of extrabody probes are used EM signals are propagated through all the diameter of the body, namely via the tissues between one probe and the target area and via the tissues between one target area and at least one different probe. This is different from using the implantable intrabody element 101 that allows propagated EM signals only via the tissues between the target area and the extrabody probe 102. intrabody element 101 Each tissue is a dielectric layer that introduces attenuation and/or dispersion to EM radiation that is transmitted via the body. For example, the skin layer and/or the fat layer, generates a return signal that causes large interference to the receiving probe, for example when the interfering return signal is larger than the desired return signal from the monitored tissue. Therefore, having the implantable intrabody probe 101 in proximity to a monitored tissue reduces or eliminates such interferences

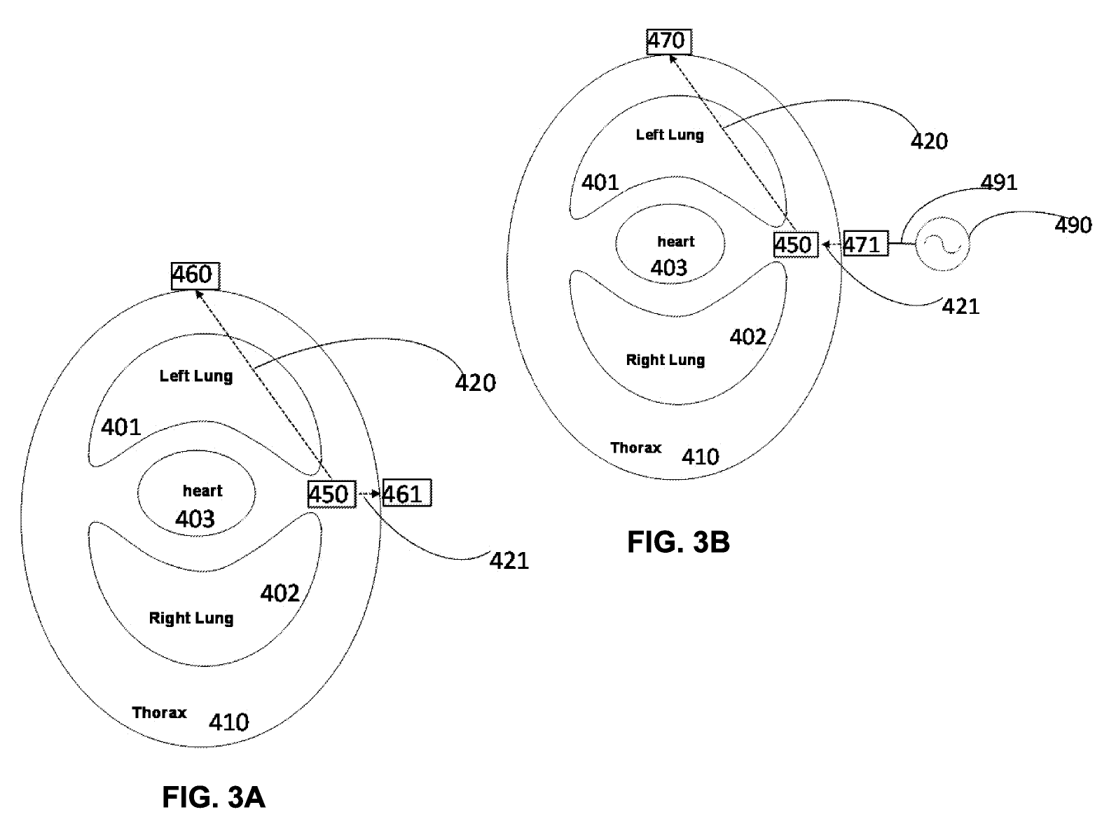

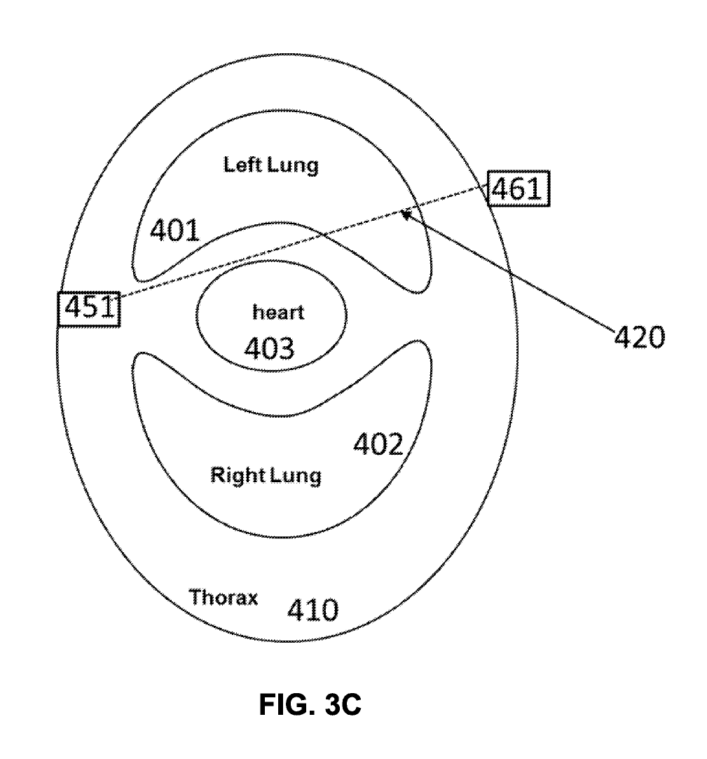

As described above, the system may be used for monitoring thoracic tissues. For example, reference is now made to FIGS. 3A and 3B, which are schematic illustrations on an axial thoracic plane of a patient monitored by the monitoring system 100. The figures depict the left lung 401, the right lung 402 and the heart 403. In this embodiment, the implantable intrabody probe 450 in FIG. 3A is implanted within the chest cavity and directed to transmit an EM signal via the left lung 401 toward the extrabody probe 460. The directed EM signal travels through the left lung 401 as can be seen in 420, and received by the extrabody probe 460. Optionally, the extrabody probe 460 performs the aforementioned analysis and/or the measuring of the EM signal to calculate one or more desired biological parameters. As descried above, the implanting of the intrabody probe 450 within the chest cavity allows avoiding some interferences and/or additional attenuations and/or dispersion which could have been induced if the EM signal would have been transmitted from the back of the patient.

In FIG. 3C, the intrabody probe 451 is implanted subcutaneously or under the fat layer and above the muscle layer on the back of the patient. In this embodiment, the intrabody probe 451 is adapted to transmit an EM signal into the left lung 401, and the extrabody probe 461 is adapted to receive the EM signal. The EM signal travels through the left lung 401 as can be seen in 420. The extrabody probe 461 may process and/or measure the EM signal, for example as described above.

As the intrabody element 101 is placed beneath the fat layer and above the muscle, it allows better directing the energy of the EM signal in a desired direction. This is achieved because of the difference between the dielectric properties of the fat layer and the dielectric properties of the muscle layer deflects EM radiation that travels from the fat layer to the muscle layer, so that less or no EM radiation is not transferred to the receiving probe and/or transmitted to the region of interest. Energy penetrating through multiple dielectric layers for example of skin-fat-muscle-lung scatter more perpendicularly than energy coupled into muscle-lung multilayer structure, inter alia as the EM energy is more easily coupled to high dielectric coefficient materials such as the muscle than compared to penetrating through low dielectric materials such as fat. The intrabody probe 451, which is implanted beneath the fat layer and/or above the muscle tissue, alleviates the dielectric properties difference and radiates directly into the muscle tissue, bypassing the fat layer. Such probe can be matched to the muscle and therefore be reduced in size as well.

It should be noted that the energy consumption of the monitoring system 100 is relatively low in relation to the energy consumption of a system that includes only extrabody probes. In such a manner, the specific absorption rate (SAR) of the target area and neighboring tissues is reduced. This is achieved as there is less need to transmit high power level to penetrate some skin, fat and/or muscle layers which are penetrated when only extrabody probes are used.

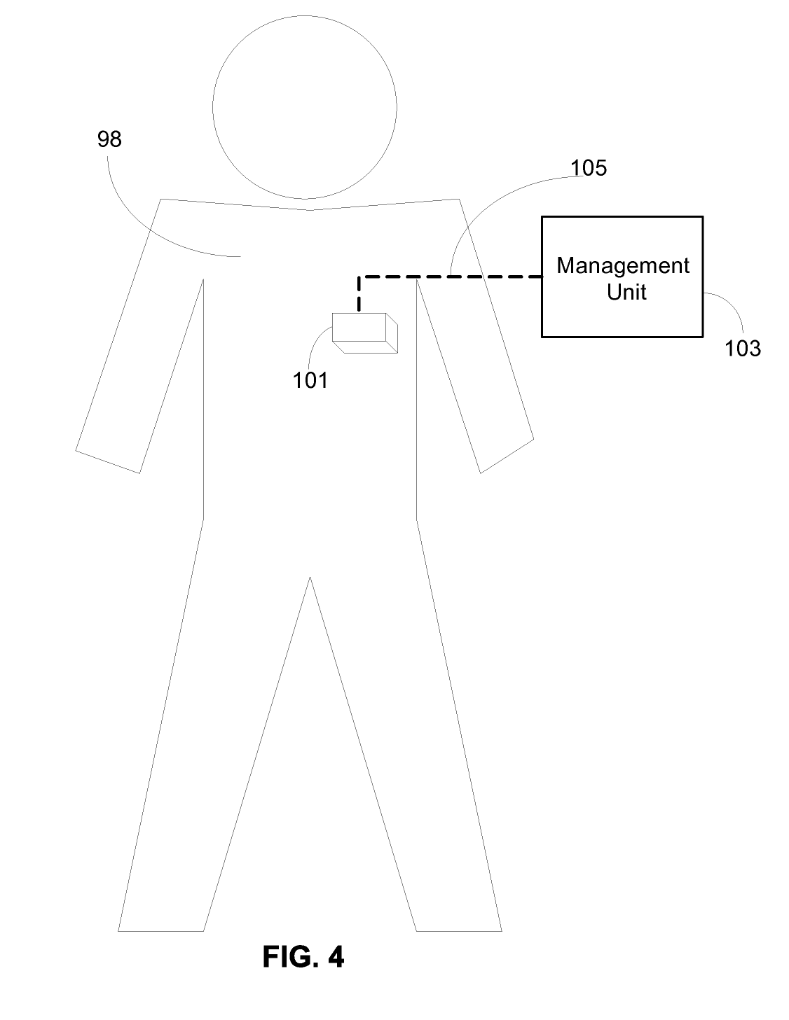

According to some embodiments of the present invention, from example as depicted in FIG. 4, the intrabody element 101 transmits data pertaining to the measurements directly to a patient management unit (not via the extrabody probe) via connection 105 optionally via the communication interface 208 described below and in FIG. 5. In such an embodiment, the at least one intrabody element 101 transmits the EM signal into an internal body part or an organ of patient 101, and adapted to receive the EM radiation returned and/or reflected from the internal body part or organ. Said at least one intrabody element 101 may receive the EM radiation reflected from the body by the same sensor that transmitted the EM radiation, or may receive the EM radiation reflected from the internal body part by an element different from the element that transmitted the EM radiation. Said different element may be an element located on the intrabody element 101 that transmitted the EM radiation, or in another intrabody probe (not shown in the figure). Such an embodiment may be used to increase the adherence of patients to use the monitoring apparatus, and to measure themselves, as in this embodiment the measurement may take place without the active involvement of the patient, by taking short measurements, for example a few milliseconds measurements every few minutes, and/or hours and/or days and/or weeks and/or when detecting a posture. The measurements or the processed data may be sent to an extrabody probe that may be attached and/or a non-attached, for example placed on a wall in the patients home or integrated into a chair, so that the monitored patient has just to stand next to it, and the intrabody probe will communicate the data, for example said measurements, to the extrabody probe.

In an embodiment where the intrabody probe is adapted to send the EM radiation and receive the reflected EM radiation from the internal body tissue, the disturbance and/or interference received by the sensor is reduced, since the sensor has less dielectric layers separating it from the region of interest. Each said dielectric layer, for example skin layer or fat layer, or muscle layer introduces interference and/or attenuation and/or dispersion for example due to dielectric inconsistency, where in the case of an externally placed probe that sends and receives the EM radiation, the encounter of the EM radiation with the body, for example with the skin layer and/or the fat layer, generates a return signal that causes large interference, for example in the magnitude of about 10% of the radiated power, to the receiving sensor from for example immediate dielectric layers. Said return signal from the skin and/or fat layer has substantially larger power than the returned signal from the area of interest. Therefore, having an intrabody probe that is close to the monitored tissue alleviates this interference, and may also alleviate the change of said interference over time, due for example skin elasticity or due to soft skin, as the intrabody probe is closely integrated to the body: such close integration to the body, for example attaching to the muscle or to the rib cage, may cause fewer changes over time of the returned interfering signal from immediate dielectric layers, thus this enables to reduce such interference by for example acquiring a baseline and monitoring changes of such baseline over time. In an embodiment where the intrabody probe's radiating element's interface with the internal tissue, for example the muscle layer, has a dielectric material that is approximately similar to the dielectric coefficient of the internal tissue, than such interference is even more minimized and the returned signal from the area of interest is also enhanced due to closer proximity of the intrabody element to the area of interest. Moreover, also the said changes over time of the interfering returned signal are reduced as the intrabody element may be less affected by movements of the skin and/or fat layer.

It may be beneficial to reevaluate and/or estimate the amplitude and/or phase, especially for narrowband signals, of the transmitted signal for the measurement of the biological parameter. It is beneficial as the calculation of the monitored biological parameter is based on a difference between the transmitted and received signals, where said difference which may be expressed by phase and/or amplitude difference, enables the calculation of the dielectric related properties. Such revaluation may be performed according to one or more of the following:

1. Amplitude revaluation—may be locally performed at the intrabody element 101 using an amplitude detector, for example a power-meter, that samples the transmitted amplitude and digitally sends it to the extrabody probe 102 that uses reevaluate the transmitted amplitude accordingly. The amplitude detector may be a part of the intrabody element 101 or implanted to communicate therewith. The amplitude detector, as the IMD above, may share resources with the intrabody element 101.
2. Phase revaluation—may be achieved by using differential measurements from two or more extrabody detectors, for example attached sensors, as depicted in 460 and 461 in FIG. 3A. One of the extrabody probes, 461 is placed close to the intrabody probe 450 which is subcutaneously or under-fat-layer implanted in the patient. The intrabody probe 450 sends two signals to the extrabody detectors, for example signal 420 towards extrabody probe 460 and signal 421 towards extrabody probe 461. Because extrabody probe 461 is close to intrabody probe 450, and there is small distance with little variation of dielectric related parameters over time of the skin and/or fat layers that separate extrabody detectors 450 and 461, one can assume that the signal travels from extrabody detector 450 to extrabody detector 461, for example as shown in 421 changes very little over time.

Reference is now made to a mathematical description. X₁ denotes the phase of the transmitted signal from intrabody probe 450 at time t₁, and X₂ denotes the phase of the transmitted signal from intrabody probe 450 at time t₂. Similarly, Y₁ denotes the phase of the received signal at probe 461 at time t₁ and Y₂ denotes the phase of the received signal at probe 461 at time t₂. Similarly, Z₁ denotes the phase of the received signal at probe 460 at time t₁ and Z₂ denotes the phase of the received signal at probe 460 at time t₂.

Since, as explained above signal 421 changes very little over time, it is understood that Y₁−X₁=Y₂−X₂ So, X₁−X₂=Y₁−Y₂ and because Y₁−Y₂ is known as it is measured, the value of X₁−X₂ is received.

T denotes a trend of the calculated and/or measured biological parameter, for example as calculated based on the changes of the dielectric related properties. Such dielectric related properties change may be measured by looking at the changes of the differential signal measured between the transmitting probe 450 and the receiving probe 460, namely the changes of Z(t)−X(t).

At t₁, the differential signal measured between the transmitting probe 450 and the receiving probe 460 is: T₁=Z₁−X₁. Similarly, at time t₂, the differential signal measured between the transmitting probe 450 and the receiving probe 460 is: Z₂−X₂

The trend T₁₂ between time instances t₁ and t₂ is:

T₁₂=T₁−T₂=Z₁−Z₂−(X₁−X₂)

but as shown above:

X₁−X₂=Y₁−Y₂

Since Y₁−Y₂ and Z₁−Z₂ are known as they are measured at the probes 463 and 460 at time instances t₁ and t₂, the trend T₁₂ may be calculated so that measures the change of the biological parameter between time instances t₁ and t₂;

T₁₂=T₁−T₂=Z₁−Z₂−(Y₁−Y₂)

In light of the above, the trend of the phase Z over time may be calculated. It is noted that external elements 460 and 461 may be connected to each other, optionally via a cable,

Using a similar concept and configuration, differential amplitude and/or delay and/or other EM related signal may be calculated and used to monitor the biological parameter.
3. A phased locked loop (PLL) phase revaluation—phase revaluation may be also achieved by using a PLL that may be part of the intrabody probe 450 or connected thereto. As shown in FIG. 3B, a signal generator 490 transmits a low frequency signal, for example 10 MHz towards an extrabody probe 471 via for example cable 491. The extrabody probe 471 may transmit the directed EM signal towards the intrabody probe 450, for example as shown in 421. The extrabody probe 471 is optionally placed in proximity to the intrabody probe 450 which is subcutaneously or under-fat-layer implanted in the patient. Because extrabody probe 471 is close to intrabody probe 450, and there is small distance with little variation of dielectric related parameters over time of the skin and/or fat layers that separate the probes 450 and 470, one can assume that the signal travels from probe 471 to probe 450, for example as shown in 421 changes very little over time, and is introduced to a relatively limited delay. Such signal between the external and internal probes can be induced by one into the other using a magnetic field for example by using magnetic loop or coils. Since the body permeability is close to 1, the magnetic field will penetrate the body in approximately the speed of light and will not be affected by changes in permittivity coefficient changes and small geometrical (distance) changes between the internal and external probes.

The intrabody probe 450, by using a PLL that is a connected thereto or a part thereof, transfers the signal into a high frequency signal, for example 1 GHz, that is synchronized with the phase of the signal transmitted to it. The intrabody probe 450 may transmit towards the extrabody probe 470, through the area of interest, for example signal 420 through the left lung 401, or transmit to more than one external sensor (not shown in the Figure). By using differentiated measurements, in for example two time instances using the signals of external probes 470 and 471 as explained above, one can derive the transmitted phase of the signal that the intrabody probe 450 transmits and/or derive the phase and/or amplitude trend over time of signal 420 received by extrabody probe 470. Such trend enables to calculate the change of the monitored biological parameter over time. It is important to note that in such embodiment the phases of signals of the extrabody probe 470 and of the intrabody probe 450 are locked together. So, similarly to what described here, there may be another embodiment where intrabody probe 450 may comprise a free running oscillator as known in the art which is used for transmitting the EM radiation, and the intrabody probe 450 transmits the EM signal towards extrabody probe 471. The extrabody probe 471, which is not connected in this embodiment to the signal generator 490, receives such transmission and may lock its phase accordingly. This enables to use this phase lock to derive the transmitted phase of internal element 450 by external element 470, by differential measurements for example as explained here above. The PLL is one way to describe a component which generates a signal based on a reference signal, where the signal generated can be a regeneration of the reference signal or any derivative of that signal, like higher or lower frequency which are multiplication of the reference signal by a rational number, or any other manipulation of the reference signal.

It is noted that external elements 470 and 471 may be connected to each other, optionally via a cable.

According to some embodiments of the present invention, the intrabody element 101 comprises a reflector that is set to reflect EM signals that irradiates it, for example from the extrabody probe 102. The reflector interacts with and/or affects the EM signals which are delivered to the intrabody element 101. The reflector is optionally includes one or more elements made of a conductive material, for example antennas, conductive plates and/or the like. Such elements may be used for directing, reflecting, focusing, and/or dispersing the EM signal in the body in certain directions, optionally set in advance. Optionally, the intrabody element 101, with the reflector, is positioned next to or in proximity to the tissue or organ that is to be monitored. For example, in case the monitored tissue is the lung, the intrabody element 101 is implanted so that the reflector is at the lower portion of the pleural cavity, above the diaphragm or in the case where the monitored tissue is a tumor in the abdomen the reflector is inserted on the side of the tumor opposite the external probe. Optionally, the extrabody probe 102 transmits an EM signal and receives the reflection thereof. In another embodiment, an extrabody transmitting unit, which is positioned on the thorax of the patient, for example above the diaphragm, transmits an EM signal that is reflected and/or steered by the reflector, to an extrabody receiving unit that is externally positioned on a different location, such as on the lateral side of the thorax of the patient. Such steering and/or focus and/or directing of the EM energy by the intrabody probe may be done for example by placing the intrabody probe, for example such reflector, in a certain angle in relation to the sending and receiving extrabody probes so that it can reflect the EM radiation from one extrabody probe to the other extrabody probe. Another example of focusing the EM radiation may be by using a concave reflector that focuses the EM radiation in a certain direction. Such concavity may also enable a gain for the reflected signal as known in the art. Optionally, the reflector comprises a non-linear component, such as a mixer, that is adapted to generate inter-modulations of two different frequencies received thereon or multiplication of one frequency to itself. The mixer may be adapted to altering to frequency of an EM signal, for example, in case the extrabody probe 102 transmits EM energy in two frequencies, for example at about 912 MHz and about 910 MHz. The reflector may reflect the received a mixture of the transmitted EM energy signal in a frequency of 914 MHz which is an intermodulation of the two received signals frequencies. Optionally, the reflector comprises a frequency changing active element that changes the frequency of the received EM signal in order to avoid interference between the transmitted and reflected signals. Optionally, the reflector may be angled to direct the transmitted EM radiation to pass via a certain path, toward the extrabody probe 102 or a receiving unit. The extrabody probe 102, or a receiving unit, is optionally positioned according to upfront knowledge of the location of the implanted reflector of the specific patient. Optionally, such kind of reflector may be an active element which intensifies the intercepted EM radiation signal, regenerates the signal, and/or manipulates the signal. Optional manipulation of such signal may include modulating the signal, demodulating the signal, changing a spread spectrum code of the received signal and shifting phase and/or frequency and/or amplitude.

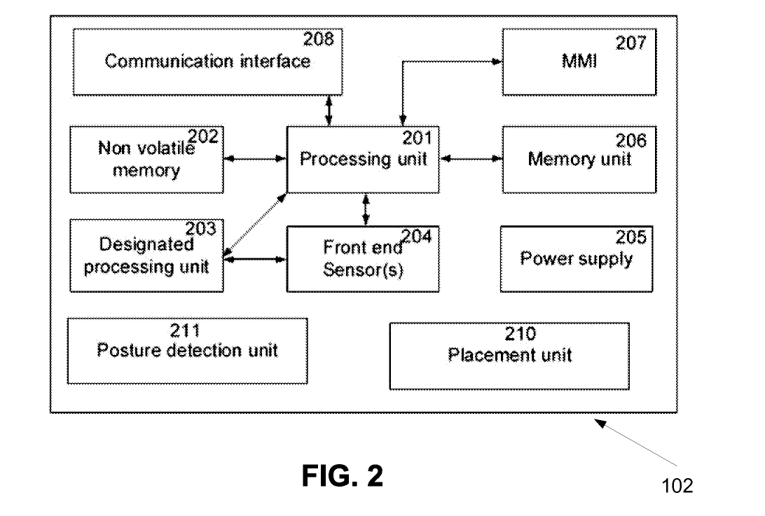

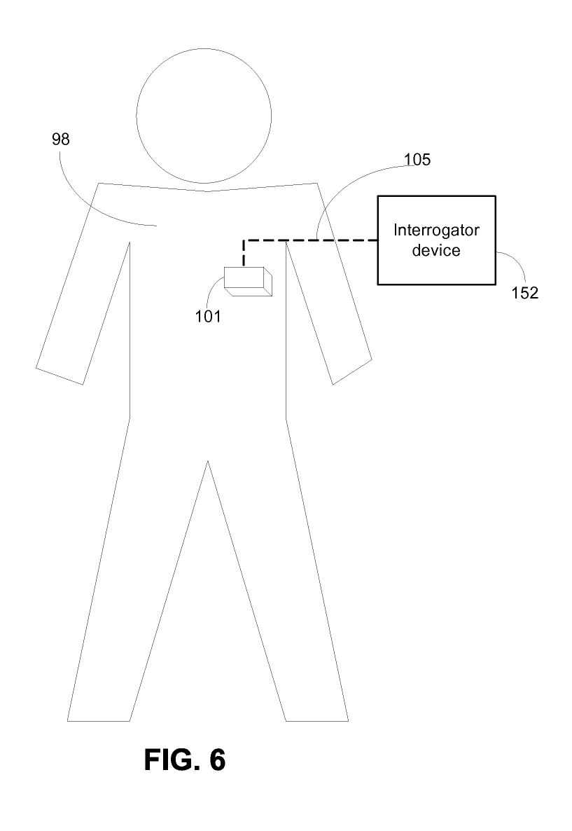

For example, reference is now made to FIG. 2, which is a schematic illustration of a set of components 200, some optional, of an exemplary extrabody probe 102 and to FIG. 6, which is a schematic illustration of a set of components, some optional, of an exemplary intrabody element 101, according to some embodiments of the present invention. These components may be as described in international patent applications No. WO2009/031149 and No. WO2009/031150, which are incorporated herein by reference. The power supply element circuitry 205 of the exemplary extrabody probe 102 may be used to energize the exemplary intrabody element 101. The power supply element circuitry 205 is charged by an energy storage device, a battery, optionally rechargeable. Optionally, the intrabody element 101 is energized from the outside of the body using electrical induction. For example by using an energy coupler and/or induction coils for receiving external electromagnetic signals, for example an RF magnetic field and converting them into electrical energy for recharging the energy storage component or directly supplying energy to the intrabody probe's components. Other alternative may be to supply energy by using ultrasonic coupling and/or vibrations and/or piezoelectric transducers for energizing the device with a remote ultrasonic energy source. Optionally, the intrabody element 101 may be energized by induction of a low frequency magnetic field, for example 13.4 MHz, from a source located outside the body, in an element which may be dissociated from the extrabody probe 102 or employed by it. Such a low frequency magnetic field has a good penetration to the body. The power supply element circuitry converts the magnetic field into electrical field, as known in the art, for example by an implanted coil that may be a part of the intrabody element 101, which may rectify the received electromagnetic signal to create a direct voltage, which may be used as a power source for the power supply element circuitry or charge a battery and/or capacitor for a later use. Optionally, the intrabody element 101 may contain a power source that is rechargeable from the outside of the body by means of energy transfer through the skin of the patient.

For example, the power supply element circuitry 205 optionally includes an induction charger to charge the power supply element circuitry 2005 of the intrabody element 101 that uses an energy coupler and/or induction coils for receiving external charging energy, for example a magnetic field, from the extrabody surroundings. The power supply element circuitry 2005 converts the received energy into electrical energy for energizing and/or charging an implanted energy storage component.

Additionally or alternatively, the intrabody element 101 may use the power source of an IMD such as a defibrillator, a syncope detector, arrhythmia device, a neurological stimulator, a Ventricular Assist Device (VAD), a neuromuscular stimulator, a pacemaker, a CRT device, a CRT-D device and others. Additionally or alternatively, power may be optically transmitted in for example infrared wavelength energy couplers for example as high-efficiency photoelectric devices. These alternatives and others are understood by a skilled artisan, and are described for example in US Patent Application 2008/0221419, METHOD AND SYSTEM FOR MONITORING A HEALTH CONDITION and international patent application WO2007/066343 IMPLANTABLE BIOSENSOR ASSEMBLY AND HEALTH MONITORING SYSTEM by Furman, U.S. Pat. No. 5,833,603 Implantable biosensing transponder by Kovacs, U.S. Pat. No. 7,686,762 Wireless device and system for monitoring physiologic parameters by Najafi, which are incorporated herein by reference.

Optionally, the extrabody probe 102 comprises a communication interface 208 for establishing and/or maintaining connection and/or exchange information and/or data, optionally bidirectional, with the intrabody element 101 and/or with a patient management unit 103, and/or with an interrogator unit 152. The connection allows the extrabody probe 102 to transfer data, for example as described above and/or data that is stored in the memory unit 206. Optionally, a similar communication unit 208, referred to herein interchangeably as a communication interface 208, is part of the intrabody probe and configured to send and/or transmit information. The communication unit 208 may share components with the transducer. Specifically the communication unit may use the same antenna used by the transducer. The communication between the intrabody element 101 and the extrabody probe 102 may be wireless communication for example by means of an RF signal, a magnetic signal, a modulated sound signal, and/or an electrical conduction signal, for example, see US Patent Application Pub. No. 2004/0011366, and other references as specified above.

Optionally, the communication interface 208 is based on a wired connection, for example a universal serial bus (USB) interface, for communicating with a patient management unit. Optionally, the communication interface 208 is used to upload state parameters, version control software elements for updating firmware and software components, and for reporting current and recorded information such as clinical parameters such as heart rate, breathing frequency, edema condition, and/or any parameter measured by one of the aforementioned probes, and any parameter or data calculated based thereupon.

As depicted in FIG. 2, the extrabody probe 102 may include one or more front-end sensors 204, such as EM transceivers, for transmitting and/or receiving a plurality of EM signals and/or pulses toward the intrabody element 101. The EM signals may be transmitted in a desired pulse and allows the capturing of a reflection thereof from various areas on the surface of the patient's body. Optionally, the transmission or reception is adjusted according a selected operational mode, for example according to a selected swept frequency, a selected frequency hopping chirp, and the like. Other modes and/or gating patterns according to which the EM signal may be transmitted and/or received are described International Patent Applications Number WO2009/031149, and WO2009/031150, which are incorporated herein by reference. Optionally, the EM signal is delivered into the thorax as a narrow bandwidth signal, although other acquisition regimes are possible such as wide bandwidth signals.

Optionally, the extrabody probe 102 includes a processing unit 201 to process and analyze the received data over the communication interface 208, and/or may be associated with an MMI 207 for a care taker, for example a display monitor that enables viewing the monitored data. Alternatively, an input/output interface such as a laptop, a tablet, a Smartphone, a PDA and the like may receive and present the data. The extrabody probe 102 may use the input/output interface to transfer, via a wire or wirelessly, the received data, to a remote client. Such communication may be established over an internet connection, a cellular connection, a Bluetooth™ connection and the like.

As described above, the intrabody element 101 and/or the extrabody probe 102 communicates with the patient management unit 103, optionally using the communication interface 208 as explained above and depicted in FIGS. 1 and 4 through connection 105 and or 110 which may be a wireless data interface. The connection may be a wireless data connection, such as an example an infrared (IR) connection, a wireless fidelity (Wi-Fi) connection, a Bluetooth™ connection, a radio connection that uses the transducer for transmitting and/or receiving the EM signal, a universal asynchronous receiver transmitter (UART) connection, and/or an radio frequency (RF) connection based on the frequency bands which are specified above.

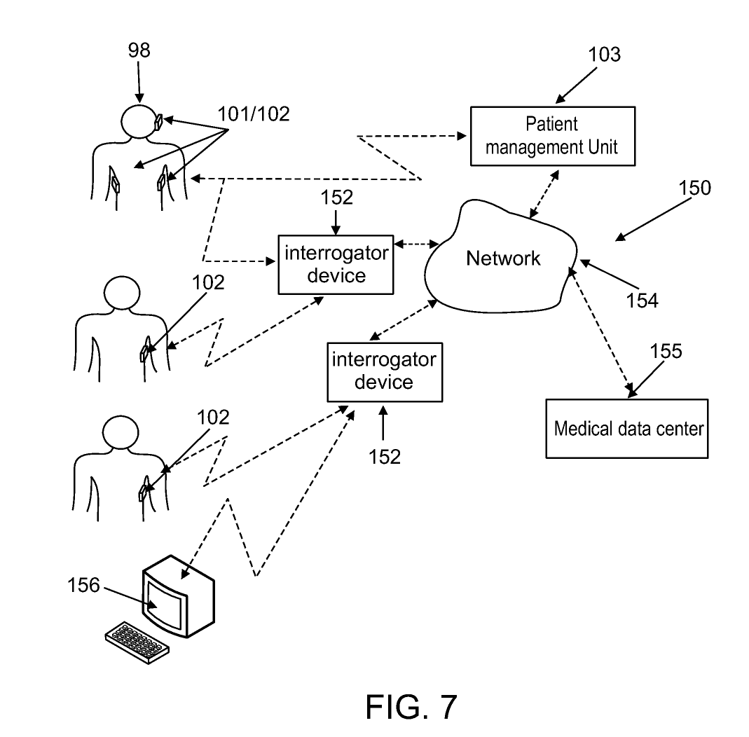

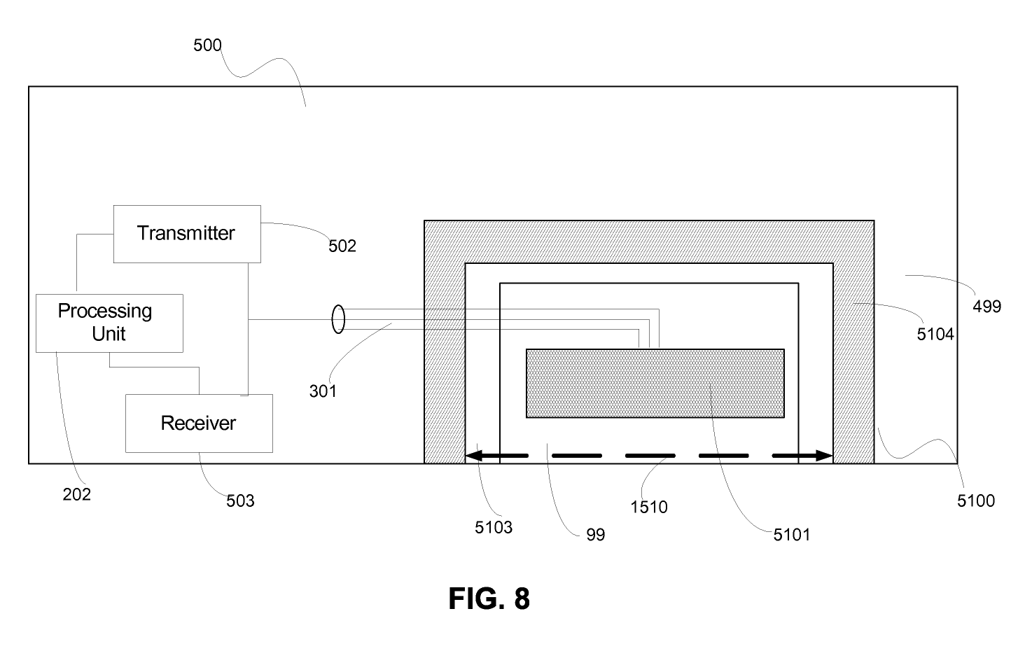

Optionally, as depicted in FIGS. 7 and 8, the intrabody element 101 may communicate with the patient management unit 103 and/or with an interrogator device 152, for example as described in International Patent Applications Number WO2009/031149, and WO2009/031150, which are incorporated herein by reference. For example, the user interrogator device 152 may be integrated into a standard hospital monitor, a third party tele-health gateway in the patient's home, and/or a Smartphone.

Optionally, the intrabody element 101 forwards, optionally periodically, gathered data pertaining to the monitored biological parameters and/or dielectric related properties and/or changes, analyzed or not, to the patient management unit 103 and/or to the interrogator device 152 that forwards the data to the patient management unit 102. The interrogator device 152 forwards, optionally periodically, instructions, updates, and/or reconfigurations from the patient management unit 103 to the monitoring system 100. Optionally, the interrogator device 152 is used to transfer energy to the intrabody element 101, for example using inductive charging methods as described above, for example by forming a low frequency magnetic field.

The components of the intrabody element 101, for example as depicted in FIG. 6, may be grouped and/or enclosed and/or sealed in a housing, for example a housing adapted for implantation in a patients body, which may be a sealed housing adapted for implantation in a patient's body. The housing may be made of a biocompatible material which minimally attenuates the EM signal, such as polyurethane. External and internal parts may be constructed from materials compatible with biological tissues suitable for long term use with no tendency to create infections, allergic or immune reactions, such as, for example, titanium, titanium alloys, and polyurethane. Furthermore, any EM radiation delivered by internal or external parts should comply with human radiation exposure regulations for example IEC standard 62209-1, which is incorporated herein by reference.

Optionally, the patient management unit 103 and/or interrogator device 152, as depicted in FIGS. 6 and 7 may communicate, optionally through network 154, with a medical data center 155, for example as described in International Patent Applications Number WO2009/031149, and WO2009/031150, which is incorporated herein by reference.

Optionally, the intrabody element 101 is an active element which intensifies, regenerates, and/or manipulates the intercepted EM signal. Optional manipulations of the intercepted EM signal include modulating the signal, demodulating the signal, and shifting phase and/or frequency and/or amplitude.

Optionally, any of the probes 101, 102 is combined with biological sensors, such as electrocardiogram (ECG) electrodes, gyroscopes, temperature sensors, and/or electromyography (EMG) sensors, ultrasound transducers, blood pressure sensors, for example ultrasonic, pulse oximeters, activity sensors, for example accelerometers and tiltmeters, microphones, capnometers, coagulometers and/or any other sensors configured for gathering data related to the physical condition of the monitored patient and/or one of its organs. As used herein, a physical condition means data related to the physical activity, vital signs, biological parameters, and/or any other medical and/or biological information which indicative of the patient wellness and/or fitness of the monitored patient. This combination enhances the ability of the system 100 to measure the biological parameters and/or the ability to perform a clinical assessment based on the biological parameters. For example, posture identification may be enhanced by using of a tiltmeter. In some embodiments of the present invention, the analysis allows calculating a biological parameter of a certain patient, such as a clinical state, based on an integrative index. For example, the biological parameter may be determined based on a combination between the dielectric related properties of an internal tissue, such as the pulmonary tissue and/or fluid content build up pace and vital signs and/or detected trends of vital signs which are acquired using one or more of the aforementioned additional sensors.

As described above, the monitoring system 100 may be suited for long term monitoring, although it may be used for short term monitoring as well. A long term monitoring means monitoring in a period during which the patient or the monitored tissue or organ undergoes one or more changes that may influence the intercepted EM signal. Such changes may be for example taking off at least one external part of the monitoring device and putting it back on, which may be referred to as sensor placement and replacement, repositioning at least one external part of the monitoring device, a misplacement or disengagement of the at least one external part of the monitoring device, different postures or activity levels of the monitored patient, movement effect, physiological processes occurring over the course of seconds, hours or days in the living body that may, possibly negatively, affect the measurement made by the monitoring system 100 and/or system, and others.

As described above, one or more of the probes of the monitoring system 100 has a sensor adapted to deliver and/or intercept an EM signal via one or more internal tissues and/or organs. The sensor includes an antenna which comprises at least one radiating element, said antenna is adapted to send and/or receive EM energy in the frequency bands as described hereunder. The antenna and/or sensor may be as described for example in International patent application pub. No. WO2009/031149, International patent application pub. No. WO2009/031150, and/or U.S. patent application No. 61/090,356, titled—ELECTROMAGNETIC EM PROBES, METHODS FOR FABRICATION THEREOF, AND SYSTEMS WHICH USE SUCH ELECTROMAGNETIC EM PROBES, which are incorporated herein by reference.

As explained, the monitoring system 100 may include multiple sensors arranged in different configurations, attached and/or non-attached, and with intrabody and/or extrabody probes. The probes may include sensors which used for both transmission and interception or exclusively for transmission or interception. Each sensor and/or probe may contain multiple transmission and/or reception elements, where it can dynamically select different configurations of receiving and transmission elements.

For example, reference is now made to FIG. 8, which is a sectional schematic illustration of a probe 500 which may be an intrabody or extrabody probe, according to some embodiments of the present invention. The probe 500 includes a housing 499, optionally made of biocompatible non conducting material with minimal attenuation to EM propagation which contains one or more of the EM sensors 5100. The dielectric property is calculated based on the reading of the EM radiation captured by the EM element. Optionally, a transmitter 502 is used to generate a signal that is transmitted to the EM element 5101 for transmission via the cable 301. Optionally, a receiver 503 is used to receive a signal that is received by the EM element 5101 via the cable 301. Optionally, the processing unit 202 is a microprocessor or any other computing unit used to analyze the outputs of the receiver 503 and/or to control the transmitter 502, and may be the processing unit described in FIG. 5. The processing is optionally performed as described in International patent application pub. No WO 2010/100649, WO2009/031149, and WO2009/031150. The front end sensor 204, as appears in FIGS. 2 and 5 and described above may be a sensor like said sensor 500, optionally comprising said transmitter 502, receiver 503 and processing unit 202.

The EM sensor 5100 optionally includes a cup shaped cavity 5103 having an opening 1510 as the depicted broken line that represents the diameter of the opening 1510, and an interior volume 99. The outer surface of the cup shaped cavity 5103, namely the external sides of the cup shaped cavity 5103 which do not face the interior volume 99 are optionally covered with one or more layers 5104 of a material for absorbing EM radiation. The one or more optional layers 104 are set to absorb electric fields and/or magnetic fields.

According to some embodiments of the present invention, the EM sensor 5100 is a printed circuit board (PCB) EM probe fabricated in known fabrication techniques, including for example as explained in U.S. patent application No. 61/090,356. The EM sensor 5100 further includes one or more emitting and/or receiving elements 5101 which are placed in the interior volume 1020. Optionally, the EM radiation is radio frequency (RF) radiation and/or microwave (MW) radiation for example from a few 100 MHz's up to a few GHz, and optionally in one or more of the frequency bands specified below. The emitting and/or receiving elements 5101 are connected, by conducting element(s) 301, such as cable(s), used to carry microwave and/or RF energy with little loss of power, to a means for generating and/or analyzing EM signals, as further described below and in the incorporated documents. The conducting element(s) 301 may be connected to the emitting and/or receiving elements 5101 via an aperture in the lateral walls of the cup shaped cavity 5103 and/or an aperture in the top wall of the cup shaped cavity 5103, for example as described application No. 61/090,356. The inner wire of the connected cable 301 is used for carrying signals intended to and/or received from the emitting and/or receiving elements 5101, for brevity referred to herein as an EM element 5101. Optionally, the EM element 5101 is driven by a coaxial cable 301 whose inner wire and shield are connected to the EM element 5101. Optionally, only the inner conductor is connected to the EM element 5101. Optionally, the shield is connected and/or coupled to the cup shaped cavity 5103. As used herein, an emitting and/or receiving element means a transducer, an antenna, for example a bowtie antenna, an ultra-wide band (UWB) antenna, a micro strip antenna, a slot fed antenna, a dipole antenna, a patch antenna, and a spiral element antenna, a feedhorn and/or a tip of a waveguide which delivers and/or collects EM radiation. Optionally the EM element 5101 contains multiple transmission and/or reception elements, and optionally multiple cables 301 are connected to it.

Said antenna is configured to send and/or receive EM energy, in several frequency bands, for example in at least one of the following frequency bands:

(a) A medical Implant Communications Service (MICS) band range, according to ITU-T Recommendation SA 1346 as implemented in the United States under the Federal Communications Commission (FCC) rules CFR47 Part 95.628 or according to the European Telecommunications Standards Institute (ETSI) Standard EN301 839, e.g. approximately 402-405 MHz.
(b) A short range device (SRD) band range e.g. approximately 862-870 MHz.
(c) a first Industrial-Scientific-Medical (ISM) band e.g. approximately 902-928 MHz. or
(d) A second Industrial-Scientific-Medical (ISM) band range e.g. approximately 2.4-2.5 GHz.
(e) One or more other frequency band ranges configured for communication between an IMD and one or more other implantable or external devices for example approximately 850-3000 MHz.

In some embodiments of the invention, the delivered EM signals comprises a multiple frequency signal, a modulated or non modulated signal, continuous or intermittent, narrow band or wide band. In some embodiments the EM radiation contains frequencies solely above 50 Mhz.

The probe 500 and/or sensor 5100 may be encapsulated, all or in part, by dielectric material that facilitates a better EM radiation coupling into the tissue that surrounds the internal part. For example, when the internal part is implanted in a muscle tissue, a dielectric substance having a relatively high dielectric coefficient is used, for example between 20 and 70, optionally approximately 50. Having a higher dielectric constant surrounding the antenna also enables to reduce the size of such antenna, as the length of the antenna changes in an inversely proportionally relation to the relative dielectric constant of the medium surrounding it. For example, as explained above, if the antenna is implanted in a muscle tissue and is surrounded by a relatively high dielectric constant, of for example approximately 50, the length of the implantable antenna in the surrounding dielectric medium decreases by roughly the inverse square root of 50, in this example, by about 7 compared to air. Optionally, the dielectric substance has a dielectric coefficient which relatively matches the dielectric coefficient of the body tissue that surrounds it. In such a manner, dielectric discontinuity is reduced and the efficiency of the transmission of the emitting element and the sensitivity of the EM receiving element is increased.

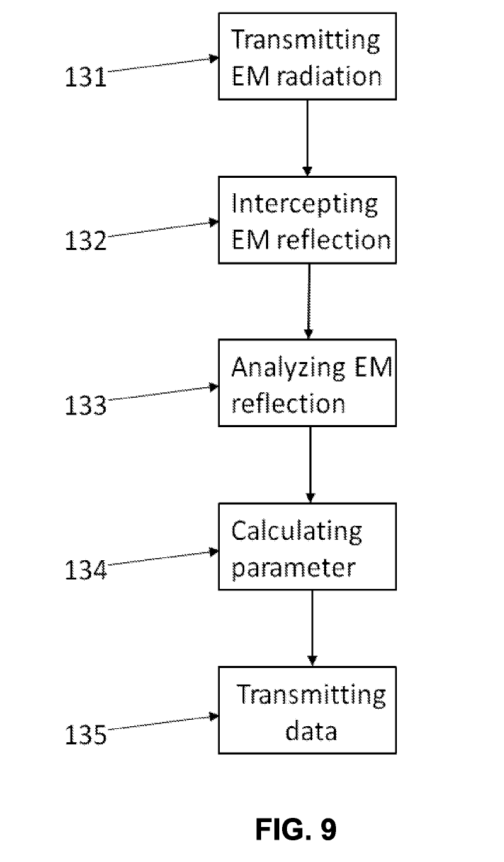

Reference is also made to FIG. 9, which is a flowchart of a method for monitoring a bodily tissue, for example thoracic tissue, using the monitoring system 100, for example as depicted in FIG. 1, according to some embodiments of the present invention. First, as shown at 131, the monitoring system 100 transmits an EM signal using a transducer and/or a sensor from an intrabody probe or extrabody probe, via a tissue or an organ of a patient. The EM signal interacts with the tissue(s) and/or organ(s). As shown at 132, at least one reflection of and/or passing through EM signal is intercepted from the monitored patient, for example after interacting with thoracic tissue of a patient in one or more radiation sessions during a monitoring period of 24 hours or more. The intercepting is optionally performed using the aforementioned one or more EM transducers, and/or by other transducers which may be part of the internal and/or external part of the monitoring system 100. As shown at 133, the reflections and/or the intercepted radiation are analyzed, for example using the processing unit that is described above. As shown at 134, the analysis allows calculating a biological parameter, for example detecting a change in the monitored thoracic tissue, for example as defined and explained above. For clarity, the analysis of the EM signal as shown at 134 may be optional, as for example the intrabody probe may intercept the reflected and/or intercepted EM radiation and analyze the reflection and/or the intercepted radiation, but it transmits the data to an extrabody probe for performance of the calculation of a parameter. Said transmission of data is shown at 135. As shown at 135, data, for example as described above, is sent, for example using a communication unit to a patient management unit or to an extrabody probe, as described above. It should be noted that the reflection and/or intercepted EM radiation may be changed as an outcome of one or more bodily movements, for example thoracic movements of the monitored patient, during the monitoring period. The affect of the one or more such movements may be compensated according to outputs of a posture detection process.

It is expected that during the life of a patent maturing from this application many relevant systems and methods will be developed and the scope of the term a detector, a sensor, a reflector, and a processing unit is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A system for monitoring at least one biological tissue of a patient during a period of at least 24 hours, comprising:
an implantable intrabody probe and an extrabody probe which propagate an electromagnetic (EM) signal, using an antenna, via at least one tissue therebetween, in a plurality of sessions during a period of at least 24 hours;
a processing unit which analyses said EM signal to detect a change in at least one biological parameter of said at least one tissue; and
an output unit which outputs said change.

2. The system of claim 1, wherein said extrabody probe delivers and intercepts said EM signal.

3. The system of claim 1, wherein said extrabody probe delivers said EM signal and said intrabody probe intercepts said EM signal.

4. The system of claim 1, wherein said processing unit analyses said EM signal to detect a change in a fluid level of said at least one tissue.

5. The system of claim 1, wherein said processing unit is part of said implantable intrabody probe, said implantable intrabody probe comprises a communication interface to transmit data pertaining to said analyzed EM signal to at least one of said extrabody probe and an extrabody patient management unit.

6. (canceled)

7. The system of claim 36, wherein said implantable intrabody probe is integrated with an implantable medical device (IMD).

8. (canceled)

9. The system of claim 36 further comprising a communication interface for receiving at least one parameter from an implantable medical device (IMD); said processing unit calculates said change according to a combination of an analysis of said EM signal and said at least one parameter.

10. The system of claim 1, further comprising a communication interface for receiving at least one parameter from an implantable medical device (IMD); and being operated according to said at least one parameter.

11. The system of claim 36, further comprising a communication interface for forwarding data related to said change to an implantable medical device (IMD) so as to allow regulation of operation of said IMD.

12. The system of claim 11, wherein said change is a cardiac ejection fraction change, and wherein said IMD is a pacemaker device, and wherein said regulation comprises adjusting the pacing parameters of a said pacemaker according to said cardiac ejection fraction change.

13. The system of claim 11, wherein said processing unit calculates cardiac output according to said change, said regulating comprises adjusting the pacing parameters of a pace making element according to said cardiac output.

14. The system of claim 11, wherein said IMD comprises a drug releasing element, said IMD adjusting the releasing pace of said drug releasing element according to said change.

15. The system of claim 11, wherein said change is a rate of change of the pressure in the heart of the patient, said IMD adjusting the pacing parameters of a pace making element according to said rate.

16. The system of claim 1, wherein said implantable intrabody probe comprises an active element which performs at least one of intensifying, regenerating, and manipulating the EM signal.

17. The system of claim 36, wherein at least one of said intrabody and extrabody probes comprises an additional sensor for gathering data related to the physical condition of the patient, said processing unit combines said change and said gathered data to determine a biological parameter.

18. The system of claim 36, further comprising a communication interface for receiving pressure value from a pulmonary arterial pressure (PAP) device implanted into said patient; said processing unit calculates said change according to a combination of an analysis of said EM signal and said pressure value.

19. (canceled)

20. (canceled)

21. (canceled)

22. (canceled)

23. (canceled)

24. The system of claim 1, wherein said EM signal is a radio frequency (RF) signal.

25. (canceled)

26. The system of claim 1, wherein said intrabody probe transmits said EM signal; further comprising an amplitude detector for sampling the amplitude of said EM signal and a communication interface for transmitting said sampled amplitude to said extrabody probe.

27. (canceled)

28. (canceled)

29. (canceled)

30. The system of claim 36, wherein said intrabody probe comprises an inductive coil for receiving an RF magnetic field for supplying energy to the intrabody probe's components.

31. The system of claim 30, wherein said extrabody probe comprises a power supply element circuitry for generating a inductive charging field.

32. (canceled)

33. (canceled)

34. The method of claim 42, wherein said implantable intrabody probe is implanted between a muscle layer and a fat layer of the patient.

35. The method of claim 42, wherein said implantable intrabody probe is implanted within the chest cavity and directed to transmit said EM signal via the lung toward said extrabody probe.

36. A system for monitoring at least one biological tissue of a patient, comprising an implantable intrabody probe, the implantable intrabody probe comprising:
a transmitter which propagates an electromagnetic (EM) signal, using an antenna, via at least one tissue;
a receiver which intercepts a reflection of said EM signal from said at least one tissue;
a processing unit which analyses said reflection to detect a change in at least one biological parameter of said at least one tissue; and
a communication unit which transfers a message, based on said change, to an extrabody unit.

37. (canceled)

38. (canceled)

39. (canceled)

40. (canceled)

41. (canceled)

42. A method for monitoring at least one biological tissue of a patient, comprising:
implanting an implantable intrabody probe in an intrabody area of a patient;
propagating an electromagnetic (EM) signal via at least one tissue;
intercepting EM signal;
analyzing said propagated EM signal to detect a change in at least one biological parameter of said at least one tissue; and
wirelessly transferring a message, based on said change, to an extrabody unit.

43. The method of claim 42, wherein said analyzing comprises analyzing said EM signal to detect a change in a fluid level of said at least one tissue.

44. The method of claim 42, wherein said EM signal is a radio frequency (RF) signal.
